(19) United States
(12) Patent Application Publication
(10) Pub. No.: US 2017/0008975 A1
(43) Pub. Date: Jan. 12, 2017

(54) Anti-Tissue Inhibitor of Metalloproteinase-4 (Timp-4) Antibodies and Methods of Use Thereof

(71) Applicant: LANKENAU INSTITUTE FOR MEDICAL RESEARCH, Wynnewood, PA (US)

(72) Inventors: U. Margaretha Wallon, Ardmore, PA (US); George C. Prendergast, Penn Valley, PA (US)

(21) Appl. No.: 15/114,272

(22) PCT Filed: Jan. 28, 2015
(86) PCT No.: PCT/US2015/013304

§ 371 Date: Jul. 26, 2016

Related U.S. Application Data
(60) Provisional application No. 61/932,459, filed on Jan. 28, 2014.

(51) Int. Cl. C07K 16/40; G01N 33/574; A61K 31/704; A61K 39/395; A61K 45/06

(52) U.S. Cl. CPC C07K 16/40; A61K 39/3955; A61K 45/06; A61K 31/704; G01N 33/57415; G01N 33/57484; A61K 2039/505

(57) ABSTRACT

Compositions and methods for treating diseases and disorders expressing tissue inhibitor of metalloproteinase-4 (TIMP-4) are provided.

This application claims the priority of U.S. Provisional Application Ser. No. 61/932,459, filed Jan. 28, 2014. The entire disclosure of the aforementioned application is incorporated herein by reference.

### FIELD OF THE INVENTION

The present invention relates to the field of cancer treatment. Specifically, compositions and methods for treating cancers expressing tissue inhibitor of metalloproteinase-4 (TIMP-4) are disclosed.

### BACKGROUND OF THE INVENTION

Several publications and patent documents are cited throughout the specification in order to describe the state of the art to which this invention pertains.

Each of these citations is incorporated herein by reference as though set forth in full.

Key parameters in the clinical management of breast cancer are the expression of steroid hormone receptors (estrogen and progesterone receptors; ER and PR) along with HER-2 overexpression and/or gene amplification. The development of effective targeted therapies for these markers has greatly improved the care and disease-free survival for these groups of breast cancer patients (Di Cosimo et al. (2010) Nat. Rev. Clin. Oncol., 7:139-47). In contrast, triple-negative breast cancer (TNBC)—as defined by the lack of ER, PR and HER-2—is a more aggressive disease subtype associated with higher risks of early recurrence and metastatic progression and limited treatment options (Dent et al., (2007) Clin. Cancer Res., 13:4429-34; Elias, A. D. (2010) Am. J. Clin. Oncol., 33:637-45). TNBC has a relapse pattern that differs sharply from hormone-positive breast cancers. The risk of relapse is much higher for the first 3 years, but then drops below that of hormone-positive breast cancers around 5 years post-treatment. TNBC is also more prevalent in younger women (<50 years) and in women of African and Hispanic descent (Turner et al. (2010) Oncogene, 29:2013-23). Lastly, women with TNBC of all stages have a five-year survival rate of 77%, compared to women with other subtypes who experience a five-year survival rate of 93% (van der Hage et al. (2011) Breast Cancer Res., 13:R68). In summary, TNBC is an aggressive subtype of breast cancer with an unmet need for effective management.

Ovarian, brain, and esophageal cancers are additional aggressive malignancies with unmet patient need in the diagnosis, prognosis and effective treatment of disease. There is a great need for identification of effective biomarkers and therapeutic targets (i.e., theranostics) that are able to diagnose, classify, prognose and treat disease (Shah et al. (2013) Cancer Epidemiol. Biomarkers Prev., 22:1185-209; Kobel et al. (2013) Cancer Epidemiol Biomarkers Prev., 22:1677-86). In summary, useful markers that are sensitive and accurate for the detection of disease by blood or tissue biopsies in brain, breast, esophageal, and ovarian cancers constitute an unmet need for effective clinical management.

### SUMMARY OF THE INVENTION

In accordance with the present invention, novel antibody molecules and fragments thereof immunologically specific for TIMP-4 are provided. Compositions comprising the antibody molecules and at least one carrier (e.g., a pharmaceutically acceptable carrier) are also provided.

According to another aspect of the invention, compositions and methods for detecting, diagnosing, treating, inhibiting, and/or preventing a disease or disorder characterized by aberrant TIMP-4 levels (e.g., due to gene arrangement, gene expression, stability, and/or modifications) are provided. In a particular embodiment, the disease or disorder is cancer (particularly breast, brain, ovarian, and/or esophageal cancer), osteoarthritis, or cardiovascular disease. In a particular embodiment, the cancer over-expresses TIMP-4 and/or has aberrant TIMP-4 activity/function (e.g., overactivity). The methods comprise administering a therapeutically effective amount of the anti-TIMP-4 antibodies of the invention to the subject (e.g., as part of a pharmaceutical preparation). In particular embodiments of the invention, the cancer is triple negative breast cancer, brain cancer, ovarian cancer (e.g., serous ovarian cancer), or esophageal carcinoma. The anti-TIMP-4 antibodies may be administered to a subject in combination with, prior to, and/or after administration of another cancer therapy such the administration of a chemotherapeutic agent, biological agents, radiation therapy, and/or surgery (resection).

### BRIEF DESCRIPTIONS OF THE DRAWING

FIG. 1A provides a Kaplin-Meier graph for TNBC patients and FIG. 1B provides a risk ratio assessment.

FIG. 2 shows the circulating levels of TIMP-4 in three TNBC patients followed prospectively by ELISA testing of blood samples from time of surgery and prior to chemotherapy.

FIG. 3 provides a schematic of TIMP-4 induced signaling. TIMP-4 binds to CD63 (1) and forms a complex with 131 integrin subunit initiating (3a) the activation of PI3K (4) and AKT(5) in a similar manner as Her-2 (2) can initiate (3b) the same pathway, resulting in survival and cell proliferation.

FIG. 4 shows the activation of Akt. TIMP-1 and TIMP-4 can activate AKT as demonstrated by Western blotting using specific pAkt^{Ser473} mAb together with pan AKTmAb (total level of AKT) and β-actin mAb loading control.

FIG. 5 shows the growth of the triple-negative human breast cancer cell-line MDA-MB-468. The presence of 2 nM TIMP-4 induces an increased growth rate that can in part be suppressed by a TIMP-4 antibody (mAb). Adding the mAb to control condition or an iso-type mAb to TIMP-4 conditions showed no effect on growth.

FIG. 6 shows the growth of MDA-MB-468 with or without TIMP-4 slow-release pellets in nude mice.

FIGS. 7A and B show the effect of modulating TIMP-4 levels in a pilot animal study. Slow-release pellets were used to elevate the TIMP-4 (FIG. 7A) levels in the mammary fatpad (mfp) prior to implanting human TNBC cells. Placebo pellets (FIG. 7B) were used for control conditions. Once tumors were established, twice weekly doses of the anti-TIMP-4 or iso-type control mAb were administered during a two-week period (i.e. in total four treatments). Animals were followed for an additional two weeks post-treatment. TIMP-4 mAb treatment had a significant effect on tumor growth in TIMP-4 elevated conditions while no effect was observed with control mAb (FIGS. 7A and 7B) or under normal TIMP-4 conditions (FIG. 7B).

FIG. 8 provides images of the immunohistochemical assessment of TIMP-4 in TNBC tumor material. Representative cores from tissue microarrays (TMAs) of patient-derived xenografts, demonstrating presence (pdx #3887 & 5471) and absence (pdx #4664 & 5166) of TIMP-4 in TNBC tumors. Similar staining pattern is obtained in patient material from an on-going prospective study of breast cancer.

### DETAILED DESCRIPTION OF THE INVENTION

Tissue inhibitor of metalloproteinase-4 (TIMP-4) has been suggested as a prognostic marker for ER-negative early-stage breast cancers (Liss et al. (2009) Am. J. Pathol., 175:940-6). TIMP-4 has also been identified as a theranostic marker for ovarian and esophageal cancers (Jiang et al. (2013) PLoS ONE 8:e76795; Maurer et al. (2014) Dis Esophagus, 27:93-100). TIMP-4 is secreted by tumor stromal cells (Pilka et al. (2006) Mol. Hum. Reprod., 12:497-503) and offers advantages as a “theranostic” molecule (i.e., both a marker and target for therapy) by being available as a target for immunotherapy.

The finding that elevated levels of TIMP-4 are associated with decreased disease-free survival may seem counterintuitive to the well-known function of TIMPs as inhibitors of matrix metalloproteinase (MMP) induced tissue degradation (Brew et al. (2000) Biochim. Biophys. Acta, 1477:267-83; Gomez et al. (1997) Eur. J. Cell Biol., 74:111-22). However, TIMPs have non-MMP associated functions in cancer, including roles in growth promotion (Denhardt et al. (1993) Pharmacol. Ther., 59:329-41; Koop et al. (1994) Cancer Res., 54:4791-7; Murphy et al. (1993) J. Cell Physiol., 157:351-8; Chirco et al. (2006) Cancer Metastasis Rev., 25:99-113), apoptosis (Bond et al. (2000) J. Biol. Chem., 275:41358-63), and angiogenesis (Nisato et al. (2005) Cancer Res., 65:9377-87) that can stimulate mammary tumorigenesis (Jiang et al. (2000) Zhonghua Wai Ke Za Zhi, 38:291-3). Moreover, clinical reports indicate that TIMPs may serve as biomarkers in various cancers. For example, TIMP-4 is an indicator of aggressive forms of glioma with rapid disease progression (Rorive et al. (2010) Mod. Pathol., 23:1418-28).

Currently, there are four known members of the mammalian TIMP family that differ in their structure, biochemical properties and expression, consistent with distinct physiological roles (Gomez et al. (1997) Eur. J. Cell Biol., 74:111-22). Among the family members, TIMP-3 is associated exclusively with extracellular matrix (Leco et al. (1994) J. Biol. Chem., 269:9352-60) whereas the others are secreted circulating proteins (Brew et al. (2000) Biochim. Biophys. Acta, 1477:267-83). TIMPs share some common features, including the formation of similar secondary structures due to formation of six highly conserved disulfide bonds that fold the protein into two domains (Williamson et al. (1990) Biochem. J., 268:267-74). The N-terminal domain, which consists of loops 1-3, is the domain that binds the active site of MMPs and blocks their enzymatic activities (Murphy et al. (1991) Biochemistry, 30:8097-102). However, attempts to model agents based on TIMP-MMP interaction, as a strategy to prevent tumor progression, had little to no effect on tumor growth and metastasis (Pavlaki et al. (2003) Cancer Metastasis Rev., 22:177-203).

Considerable research has been devoted to identifying cell surface receptors for secreted TIMPs, as part of the effort to explain their effects on cell growth, apoptosis and angiogenesis. Recently, it has been reported that TIMP-1 and TIMP-4 bind the cell surface associated tetraspanin CD63 (Rorive et al. (2010) Mod. Pathol., 23:1418-28; Jung et al. (2006) EMBO J., 25:3934-42; Pols et al. (2009) Exp. Cell Res., 315:1584-92.). Tetraspanins are membrane-associated proteins that loop through the plasma membrane four times with the N-terminus and C-terminus protruding into the cytoplasm (Mannion et al. (1996) J. Immunol., 157:2039-47).

The C-terminal domain of CD63 interacts with integrin receptors, specifically the 131 integrin subunit, through a conformational change in CD63 that takes place after binding of specific proteins to its extracellular groove (Berditchevski et al. (1997) J. Biol. Chem., 272:2595-8). TIMP-4 may be capable of binding CD63 through its C-terminal domain, thereby triggering the conformational change in CD63 which is needed to bind and activate signaling from the integrin β1-subunit, resulting in activation (phosphorylation) of AKT and the induction of a pivotal cancer-associated cell survival pathway (Jung et al. (2006) EMBO J., 25:3934-42). Taken together, TIMP-4 is multifunctional and the binding of its C-terminal domain to CD63 leading to activation of the PI3K/AKT pathway. Without being bound by theory, this activity may be sufficient to explain the pathologic relevance of the clinical observations in cancer, particularly breast cancer.

Herein, the use of TIMP-4 as a target for cancer, particularly breast, brain, ovarian, or esophageal cancer, therapy is demonstrated. In accordance with the present invention, anti-TIMP-4 antibodies and methods of use thereof are provided. Specifically, methods for inhibiting, treating, and/or preventing a disease or disorder (e.g., without limitation, cancer, osteoarthritis, or cardiovascular disease) associated with aberrant TIMP-4 expression are provided. The methods comprise the administration of at least one anti-TIMP-4 antibody to a subject in need thereof. In a particular embodiment, the cancer is breast cancer, particularly triple-negative breast cancer (lacks/negative for estrogen receptor (ER), progesterone receptor (PR), and HER-2/neu (e.g., no more than trace amounts of any of the three receptors)). In a particular embodiment, the administration of the anti-TIMP-4 antibody inhibits and/or prevents the conversion of non-invasive cancer (e.g., a non-invasive cancer such as ductal carcinoma in situ (DCIS) or a non-invasive prostate cancer such as high-grade prostatic intraepithelial neoplasia (HGPIN; Lee et al. (2006) 16:750-758) to invasive cancer (e.g., invasive breast cancer or invasive prostate cancer). In a particular embodiment, the cancer and/or the surrounding tissue aberrantly expresses TIMP-4 (e.g., increased expression compared to normal/healthy tissue). In a particular embodiment, the cancer is selected from the group consisting of, but not limited to: breast cancer, brain cancer (e.g., glioblastoma multiforme, oligodenroglioma, astrocytoma, etc.), ovarian cancer, esophageal cancer, prostate cancer, colorectal cancer, uterine cancer, seminoma, and leukemia (e.g., hairy cell leukemia).

The TIMP-4 antibodies of the invention include monoclonal and polyclonal antibodies, particularly monoclonal antibodies, or fragments thereof. In a particular embodiment, the anti-TIMP-4 antibodies of the instant invention are immunologically specific for TIMP-4, particularly human TIMP-4 (see, e.g., GenBank Accession Nos. NM_003256.2 and NP_003247.1). The antibodies of the instant invention may be humanized antibodies (e.g., humanized mouse monoclonal antibodies). In a particular embodiment, the anti-TIMP-4 antibody is clone 18:4-7, antibody fragment thereof, or derived therefrom. In a particular embodiment, the antibody of fragment thereof is selected from the group consisting of a monoclonal antibody, Fab, Fab′, F(ab′)₂, F(v), scFv, scFv₂, scFv-Fc, minibody, diabody, bispecific, and single variable domain. Composition comprising an antibody of the instant invention and at least one pharmaceutically acceptable carrier are also encompassed by the present invention.

The antibodies of the instant invention may be naturally occurring or synthetic or modified (e.g., a recombinantly generated antibody; a chimeric antibody; a bispecific antibody; a humanized antibody; and the like). The antibody may comprise at least one purification tag. In a particular embodiment, the antibody is an antibody fragment. Antibody fragments include, without limitation, immunoglobulin fragments including, without limitation: single domain (Dab; e.g., single variable light or heavy chain domain), Fab, Fab′, F(ab′)₂, and F(v); and fusions (e.g., via a linker) of these immunoglobulin fragments including, without limitation: scFv, scFv₂, scFv-Fc, minibody, diabody, triabody, and tetrabody. The antibody may also be a protein (e.g., a fusion protein) comprising at least one antibody or antibody fragment. In a particular embodiment of the instant invention, the antibody comprises an Fc region. In a particular embodiment of the instant invention, the antibody is a monoclonal antibody.

The instant invention also encompasses synthetic proteins which mimic an immunoglobulin. Examples include, without limitation, Affibody® molecules (Affibody, Bromma, Sweden), darpins (designed ankyrin repeat proteins; Kawe et al. (2006) J. Biol. Chem., 281:40252-40263), and peptabodies (Terskikh et al. (1997) PNAS 94:1663-1668).

The antibodies of the instant invention may also be conjugated/linked to other components. For example, the antibodies may be operably linked (e.g., covalently linked, optionally, through a linker) to at least one detectable agent, imaging agent, contrast agent, or therapeutic compound (see below; e.g., a chemotherapeutic agent).

The antibody molecules of the instant invention may be produced by expression of recombinant antibody fragments in host cells. Nucleic acid molecules encoding the TIMP-4 antibody fragments may be inserted into expression vectors and introduced into host cells. The resulting antibody molecules may then be isolated and purified from the expression system. The antibodies may optionally comprise a purification tag (e.g., His-tag) by which the antibody can be purified. The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

The methods of the instant invention may further comprise the administration of at least one other therapeutic for the disease or disorder being treated. For example, in the treatment of cancer, the methods may further comprise the administration of at least one chemotherapeutic agent and/or biological agent and/or anti-cancer therapy (e.g., radiation therapy and/or surgery to remove cancerous cells or a tumor (e.g., resection)). In a particular embodiment, the method further comprises the administration of an anthracycline chemotherapeutic agent. The agents administered to the subject may be contained with a composition comprising at least one pharmaceutically acceptable carrier. When more than one agent is being administered (e.g., anti-TMP-4 antibody with an additional chemotherapeutic and/or biological agent), the agents may be administered separately (before or after) and/or at the same time. The agents may be administered in the same composition or in separate compositions.

With regard to cardiovascular disease, TIMP-4 expression has been associated with an increased risk of a myocardial infarction (Weir et al. (2011) J. Cardiac Failure 17:465-471). Accordingly, the instant invention encompasses methods of inhibiting, treating, and/or preventing cardiovascular disease or heart disease. In a particular embodiment, the method comprises reducing the risk (likelihood) and/or inhibiting the occurrence of a myocardial infarction in a subject, particularly a second or further myocardial infarction. The methods comprise administering the TIMP-4 antibodies of the instant invention, optionally in a composition with a pharmaceutically acceptable carrier, to the subject (e.g., a subject having experienced a myocardial infarction). In a particular embodiment, the subject has elevated circulating levels of TIMP-4 (e.g., compared to healthy (e.g., without cardiovascular disease) subjects).

Anti-TIMP-4 antibodies have broad applications in cancer therapy. Specifically, the anti-TIMP-4 antibody molecules of the invention may be used to inhibit the growth of tumors that express TIMP-4 or that have TIMP-4 expressed in the surrounding tissue (e.g., microenvironment). The anti-TIMP-4 antibody molecules of the instant invention can be administered to a patient in need thereof, as described hereinbelow. While the anti-TIMP-4 antibody molecules of the instant invention are typically an antibody or fragment thereof, the instant invention also encompasses the use of immunotoxins wherein the anti-TIMP-4 antibody is conjugated to an agent toxic to the cancer cells such as toxins, chemotherapeutic agents, radioisotopes, radiosensitizers, and the like.

In yet another embodiment of the instant invention, the anti-TIMP-4 antibody molecules of the instant invention can be conjugated or covalently attached to another targeting agent to increase the specificity of the tumor targeting. Targeting agents can include, without limitation, antibodies, cytokines, and receptor ligands. In a particular embodiment, the targeting agent is overexpressed on the tumor as compared to normal tissue.

The antibodies as described herein will generally be administered to a patient as a pharmaceutical preparation. The term “patient” as used herein refers to human or animal subjects. These antibodies may be employed therapeutically, under the guidance of a physician for the treatment of malignant tumors and metastatic disease.

The pharmaceutical preparation comprising the antibody molecules of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The dose and dosage regimen of an antibody according to the invention that is suitable for administration to a particular patient may be determined by a physician considering the patient's age, sex, weight, general medical condition, and the specific condition and severity thereof for which the antibody is being administered. The physician may also consider the route of administration of the antibody, the pharmaceutical carrier with which the antibody may be combined, and the antibody's biological activity.

Selection of a suitable pharmaceutical preparation depends upon the method of administration chosen. For example, the antibodies of the invention may be administered by direct injection into any cancerous tissue or tumor or into the surrounding area. In this instance, a pharmaceutical preparation comprises the antibody molecules dispersed in a medium that is compatible with the cancerous tissue.

Antibodies may also be administered parenterally by injection into the blood stream (e.g., intravenous), or by subcutaneous, intramuscular or intraperitoneal injection. Pharmaceutical preparations for parenteral injection are known in the art. If parenteral injection is selected as a method for administering the antibodies, steps must be taken to ensure that sufficient amounts of the molecules reach their target cells to exert a biological effect. The lipophilicity of the antibodies, or the pharmaceutical preparation in which they are delivered, may have to be increased so that the molecules can arrive at their target locations. Furthermore, the antibodies may be delivered in a cell-targeting carrier so that sufficient numbers of molecules will reach the target cells. Methods for increasing the lipophilicity of a molecule are known in the art. If a small form of the antibody is to be administered, including but not limited to a Fab fragment, a Dab, an scFv or a diabody, it may be conjugated to a second (carrier) molecule such as, but not limited to polyethylene glycol (PEG) or an albumin-binding antibody or peptide to prolong its retention in blood.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral or parenteral. In preparing the antibody in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of anti-TIMP-4 antibody molecules may be determined by evaluating the toxicity of the antibody molecules in animal models. Various concentrations of antibody pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the antibody molecule treatment in combination with other standard anti-cancer drugs. The dosage units of anti-TIMP-4 antibody molecules may be determined individually or in combination with each anti-cancer treatment according to greater shrinkage and/or reduced growth rate of tumors.

The pharmaceutical preparation comprising the anti-TIMP-4 antibody molecules may be administered at appropriate intervals, for example, at least twice a day or more until the pathological symptoms are reduced or alleviated, after which the dosage may be reduced to a maintenance level. The appropriate interval in a particular case would normally depend on the condition of the patient.

In accordance with another aspect of the instant invention, methods of detecting an increased risk, diagnosing, or providing a prognosis for cancer or invasive potential in a subject are provided. In a particular embodiment, the cancer is triple-negative breast cancer. In certain embodiments, the method comprises detecting TIMP-4 in a biological sample obtained from a subject, wherein an increase in TIMP-4 in the biological sample compared to a corresponding biological sample in a normal subject or a subject having non-invasive cancer is indicative of an increased risk of cancer, an indication of the presence of triple-negative breast cancer, and/or an increased risk for the cancer being or becoming invasive in the test subject. Alternatively, the amount of TIMP-4 may be assessed in vivo (e.g., with in vivo imaging techniques wherein an anti-TIMP-4 antibody conjugated to a detection agent is administered to the subject). In a particular embodiment, the method comprises contacting the biological sample with at least one anti-TIMP-4 antibody, optionally conjugated to at least one detection molecule. In a particular embodiment, the biological sample is a tumor sample or biopsy, or a tissue or fluid sample obtained near the tumor. Many immunological assays are well known in the art for assaying of biological samples for the presence of a certain protein (e.g., TIMP-4) including, without limitation: immunoprecipitations, radioimmunoassays, enzyme-linked immunosorbent assays (ELISA), immunohistochemical assays, Western blot and the like. Subjects comprising the tumor and elevated TIMP-4 levels may be treated with agents to modulate the activity of TIMP-4 to normal, healthy levels (e.g., the subject may be treated with anti-TIMP-4 antibodies).

As stated above, the presence of TIMP-4 in a biological sample is indicative of the presence of cancer (e.g., triple-negative breast cancer) and indicative of invasiveness, particularly when present in quantities greater than that of normal healthy subjects. The loss of TIMP-4 in a patient, particularly one undergoing treatment, over time is indicative of remission (i.e., successful treatment). Similarly, the gain of TIMP-4 in a patient over time is indicative of recurrence. In a particular embodiment of the invention, other cancer diagnostic assays can be performed to confirm the results obtained with the instant invention.

### Definitions

The following definitions are provided to facilitate an understanding of the present invention:

The singular forms “a,” “an,” and “the” include plural referents unless the context clearly dictates otherwise.

An “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody (scFv-CH3), diabody, single variable domain (e.g., variable heavy domain, variable light domain), and bispecific. Dabs can be composed of a single variable light or heavy chain domain. In a certain embodiment of the invention, the variable light domain and/or variable heavy domain specific for TIMP-4 are inserted (engineered) into the backbone of the above mentioned antibody constructs. Methods for recombinantly producing antibodies are well-known in the art.

The term “diabodies” refers to small antibody fragments with two antigen-binding sites, which fragments comprise a heavy-chain variable domain (V_{H}) connected to a light-chain variable domain (V_{L}) on the same polypeptide chain (V_{H}-V_{L}). By using a linker that is too short to allow pairing between the two domains on the same chain, the domains are forced to pair with the complementary domains of another chain and create two antigen-binding sites.

With respect to antibodies, the term “immunologically specific” refers to antibodies that bind to one or more epitopes of a protein or compound of interest, but which do not substantially recognize and bind other molecules in a sample containing a mixed population of antigenic biological molecules. As used herein, the term “immunotoxin” refers to chimeric molecules in which antibody molecules or fragments thereof are coupled or fused (i.e., expressed as a single polypeptide or fusion protein) to toxins or their subunits. Toxins to be conjugated or fused can be derived form various sources, such as plants, bacteria, animals, and humans or be synthetic toxins (drugs), and include, without limitation, saprin, ricin, abrin, ethidium bromide, diptheria toxin, Pseudomonas exotoxin, PE40, PE38, saporin, gelonin, RNAse, protein nucleic acids (PNAs), ribosome inactivating protein (RIP), type-1 or type-2, pokeweed anti-viral protein (PAP), bryodin, momordin, and bouganin.

The term “conjugated” refers to the joining by covalent or noncovalent means of two compounds or agents of the invention.

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, and Pseudomonas exotoxin); taxanes; alkylating agents (e.g., temozolomide, nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes (e.g., cisplatin, carboplatin, tetraplatin, ormaplatin, thioplatin, satraplatin, nedaplatin, oxaliplatin, heptaplatin, iproplatin, transplatin, and lobaplatin); bioreductive alkylators such as mitomycin, procarbazine, dacarbazine and altretamine); DNA strand-breakage agents (e.g., bleomycin); topoisomerase II inhibitors (e.g., amsacrine, menogaril, amonafide, dactinomycin, daunorubicin, N,N-dibenzyl daunomycin, ellipticine, daunomycin, pyrazoloacridine, idarubicin, mitoxantrone, m-AMSA, bisantrene, doxorubicin (adriamycin), deoxydoxorubicin, etoposide (VP-16), etoposide phosphate, oxanthrazole, rubidazone, epirubicin, bleomycin, and teniposide); DNA minor groove binding agents (e.g., plicamydin); antimetabolites (e.g., folate antagonists such as methotrexate and trimetrexate); pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); anthracyclines; tubulin interactive agents (e.g., vincristine, vinblastine, and paclitaxel (Taxol®)); and antibodies (e.g., HER2 antibodies (e.g., trastuzumab)). In a particular embodiment, the chemotherapeutic agent is an anthracycline (e.g., doxorubicin, daunorubicin).

Radiation therapy refers to the use of high-energy radiation from x-rays, gamma rays, neutrons, protons and other sources to target cancer cells. Radiation may be administered externally or it may be administered using radioactive material given internally. Chemoradiation therapy combines chemotherapy and radiation therapy.

The term “biological therapy” refers to administration of biological therapeutics, typically those that work with the immune system, to destroy cancer cells and/or control side effects of other therapies. The biological therapeutic or agent may be naturally occurring in the body. Examples of biological therapeutics/agents include, without limitation: cord blood, stem cells, growth factors, cytokines, interferons, colony stimulating factors, tumor necrosis factors, interleukins, antibodies (e.g., monoclonal antibodies (e.g., herceptin)), and cancer growth inhibitors. “Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., cancer) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to halt bleeding in a subject.

As used herein, the term “subject” refers to an animal, particularly a mammal, particularly a human.

As used herein, “cardiovascular disease” refers to a disease of the heart or blood vessels. Cardiovascular disease includes, without limitation: angina, arrhythmia, coronary artery disease (CAD), coronary heart disease, cardiomyopathy, myocardial infarction, heart failure, peripheral vascular disease, artery disease, carotid artery disease, deep vein thrombosis, venous diseases, atherosclerosis, and the like.

The term “conjugated” refers to the joining by covalent or noncovalent means of two molecules or compounds of the invention. The molecules may be joined by a linker domain.

The term “linker domain” refers to a chemical moiety comprising a covalent bond or a chain of atoms that covalently attaches the targeting moiety to the cytotoxin. In a particular embodiment, the linker may contain from 0 (i.e., a bond) to about 500 atoms, about 1 to about 100 atoms, or about 1 to about 50 atoms. Exemplary linkers may comprise at least one optionally substituted; saturated or unsaturated; linear, branched or cyclic alkyl, alkenyl, or aryl group. The linker may also be a polypeptide (e.g., from about 1 to about 20 amino acids).

The term “radiosensitizer”, as used herein, is defined as a molecule administered to animals in therapeutically effective amounts to increase the sensitivity of the cells to radiation. Radiosensitizers are known to increase the sensitivity of cells to the toxic effects of radiation. Radiosensitizers include, without limitation, 2-nitroimidazole compounds, and benzotriazine dioxide compounds, halogenated pyrimidines, metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

As used herein, “diagnose” refers to detecting, classifying, and/or identifying a disease or disorder in a subject. The term may also encompass assessing or evaluating the disease or disorder status (progression, regression, stabilization, response to treatment, etc.) in a patient known to have the disease or disorder.

As used herein, the term “prognosis” refers to providing information regarding the impact of the presence of a disease or disorder (e.g., as determined by the diagnostic methods of the present invention) on a subject's future health (e.g., expected morbidity or mortality, the likelihood of getting cancer, and the risk of invasive cancer). In other words, the term “prognosis” refers to providing a prediction of the probable course and outcome of a disease/disorder or the likelihood of recovery from the disease/disorder.

Detectable labels or detection agents include, for example, chemiluminescent moieties, bioluminescent moieties, fluorescent moieties, contrast agents, radionuclides, isotopes (e.g., radioisotopes (e.g., ³H (tritium) and ¹⁴C) or stable isotopes (e.g., ²H (deuterium), ¹¹C, ¹³C, ¹⁷O and ¹⁸O), optical agents for imaging, and metals (e.g., gold). Examples of detectable labels include, without limitation, paramagnetic or superparamagnetic ions for detection by MRI imaging. Paramagnetic ions include, without limitation, Gd(III), Eu(III), Dy(III), Pr(III), Pa(IV), Mn(II), Cr(III), Co(III), Fe(III), Cu(II), Ni(II), Ti(III), and V(IV). Fluorescent agents include, without limitation, fluorescein and rhodamine and their derivatives. Optical agents include, without limitation, derivatives of phorphyrins, anthraquinones, anthrapyrazoles, perylenequinones, xanthenes, cyanines, acridines, phenoxazines and phenothiazines.

The following example provides illustrative methods of practicing the instant invention, and is not intended to limit the scope of the invention in any way.

### Example

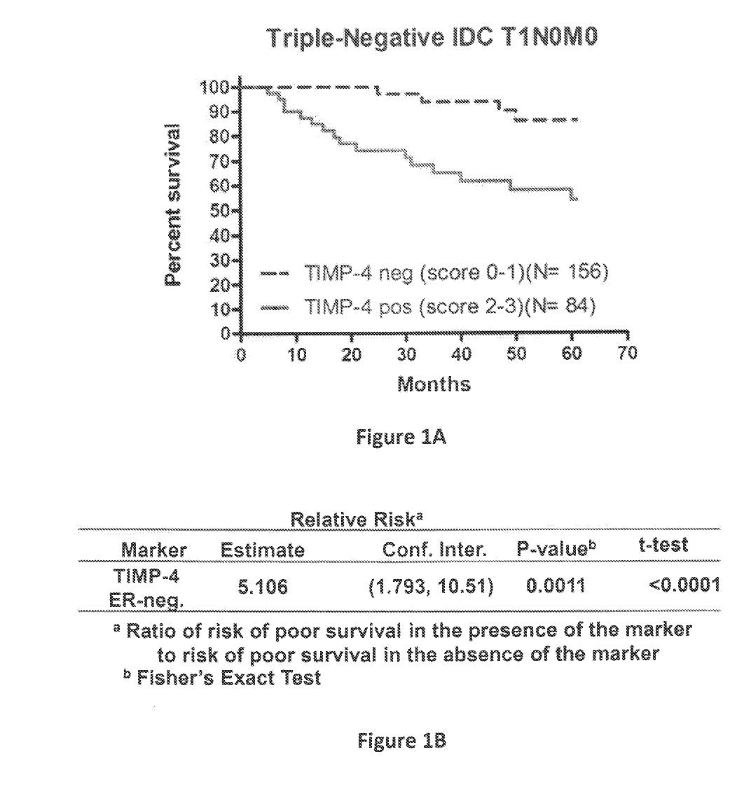

As summarized in FIG. 1, the results from a large retrospective study (N=2197) that consisted of 240 ER-negative tumor specimens established that ER-negative tumors displaying elevated levels of TIMP-4 (intermediate or strong IHC staining) are associated with shorter periods of disease-free survival, while ER-negative breast cancers with no or low levels of TIMP-4 have a better survival prognosis that differs little from ER-positive cancers (Liss et al. (2009) Am. J. Pathol., 175:940-6). The estimated relative risk for poor survival among patients with TIMP-4 positive TNBCs is 5.106. On this basis, it was determined that TIMP-4 provides a surrogate marker for the aggressive basal subtype of triple-negative breast cancers (TNBCs) that presently rely on multiple marker assessment.

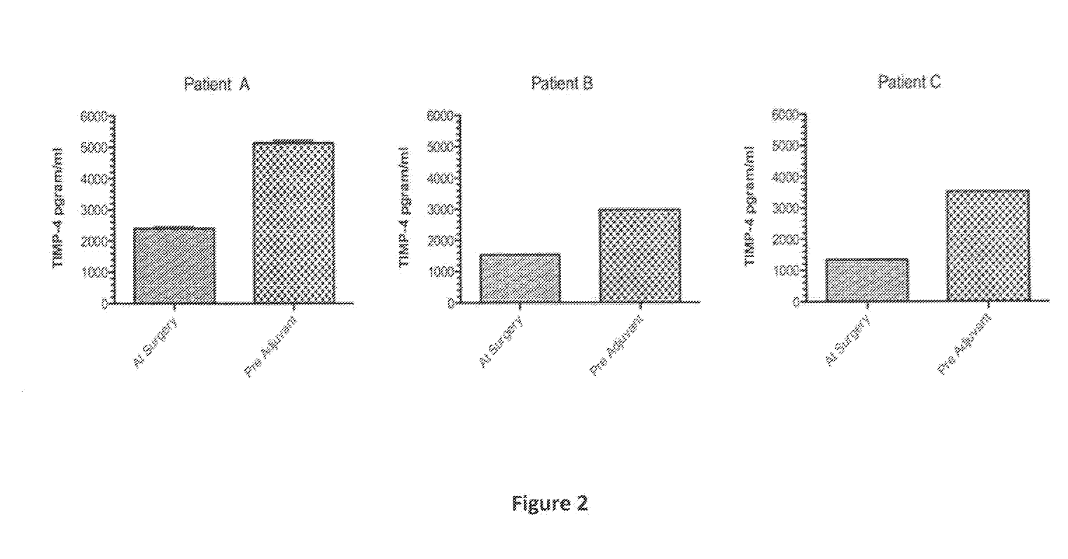

Results from the prospective study indicate similar patterns for TNBC and ER-negative cases. For the immunohistochemical analyses performed in these studies, several monoclonal antibodies (mAb) were developed with superior signal-to-noise ratio compared to a commercially available antibody (Donover et al. (2010) J. Cell. Biochem., 110:1255-61). These mAb can also be used in detecting circulating TIMP-4. TNBC patients displaying high TIMP-4 levels in the tumor microenvironment also have high circulating levels 1-3 months after tumor excision (FIG. 2). These levels may indicate an elevated risk of recurrence and/or disease progression from disseminated cells remaining after surgery. Notably, it was determined that anthracycline-based chemotherapy resulted in reduced circulating levels of TIMP-4, compared to other therapies. Enhancing the clinical significance and impact of TIMP-4 in TNBC, one can leverage the neutralizing properties of the mAb characterized herein, as a way to prevent early recurrence and spread induced by continued exposure to high levels of TIMP-4 (as seen in TNBC patients in the ongoing prospective study).

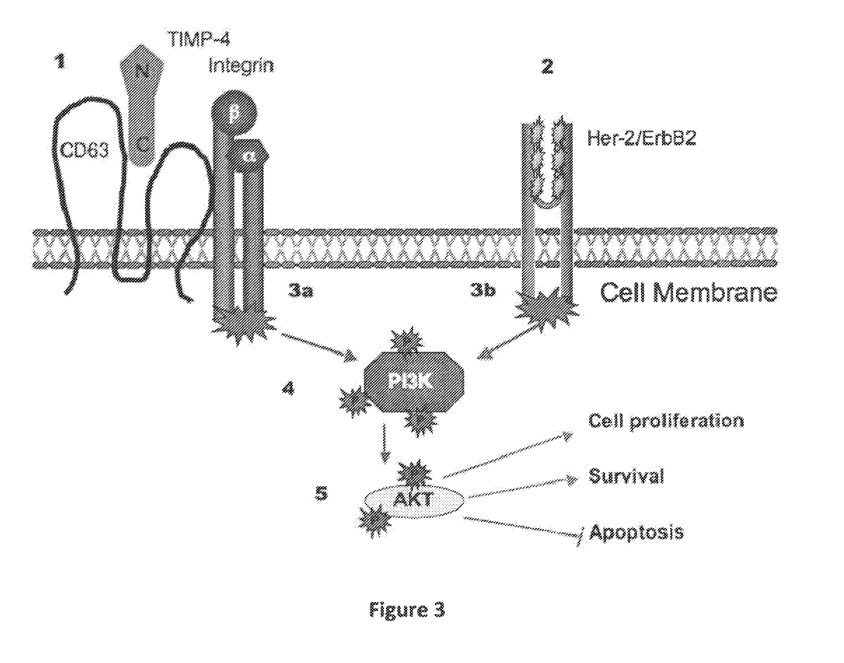

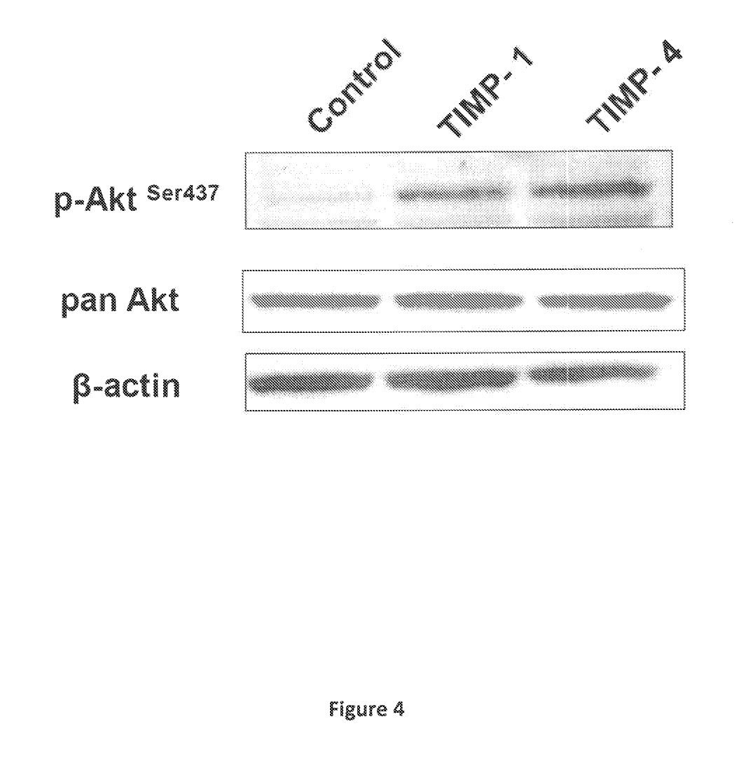

TIMP-4 appears to induce PI3K/AKT signaling, a critically important cancer cell pathway (Cantley, L. C. (2002) Science, 296:1655-7; Lopez-Knowles et al. (2010) Int. J. Cancer, 126:1121-31), through binding to the tetraspanin CD63 (Jung et al. (2006) EMBO J., 25:3934-42) (FIG. 3). In breast cancer cell cultures, TIMP-4 addition is sufficient to activate the PI3K/AKT pathway, thereby stimulating the same signaling cascade as Her-2 activation (FIG. 4). Prior to clinical use of Her-2 targeting therapy (Herceptin®), breast cancers that were positive for Her-2 were, like TNBCs, associated with fast growth rate, early recurrence and distant disease resulting in a prognosis of poor survival (Hsieh et al. (2007) Br. J. Cancer, 97:453-7).

Human breast cancer cell-lines expressing the tetraspanin CD63 (Chirco et al. (2006) Cancer Met. Rev., 25:99-113), as determined by FACS analysis, include MDA-MB-468. This cell line was used in immunoprecipitation (IP) assays and immunofluoresence staining of cells in the presence of 2nM TIMP-4, demonstrating CD63 is a functional binding partner to TIMP-4 in human breast cancer cells.

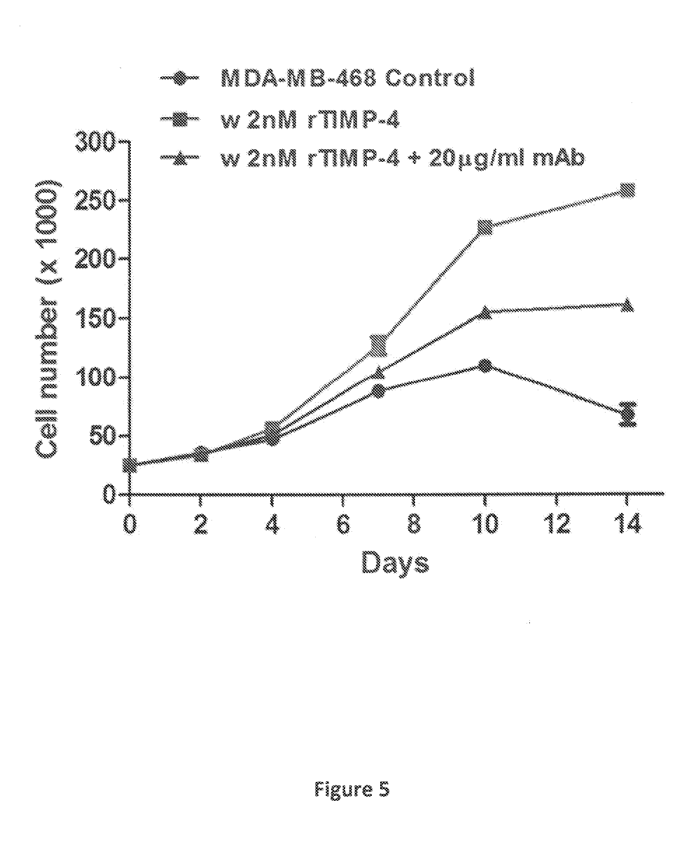

MDA-MB-468 cell were compared to the CD63 negative SK-Br-3 cells using a modified Boyden chamber to assess invasion in absence (Ctrl) and presence of 2nM TIMP-4 (N=6). The inclusion of TIMP-4 for 24 hours increased the invasiveness of MDA-MB-468 cell compared to controls, but did not increase the invasiveness of CD63 negative SK-Br-3 cells. Clonogenic growth assay (Ayene et al. (2000) Int. J. Radiat. Biol., 76:1523-1531) was used to determine the survival advantage for MDA-MB-468 cells under elevtedTIMP-4 conditions after irradiation (N=3). The presence of TIMP-4 increased survival of the MDA-MB-468 cells. This effect can be prevented by pre-treating the cells with LY294002. Human TNBC cell line MDA-MB-468 grown in normal media supplemented with TIMP-4 (R&D Systems, Inc) exhibited a relatively faster growth rate (FIG. 5). Further, cells cultured in normal medium began to detach and die as they reached contact inhibition (day 10), whereas cells in TIMP-4-supplemented media continued to survive and grow. Growth augmented by TIMP-4 could be blocked by the addition of the TIMP-4 mAb of the instant invention. Briefly, TIMP-4 mAbs were obtained by delivering a purified glutathione-S-transferase (GST) chimeric protein comprising the human full-length 224 amino acid TIMP-4 (GenBank Accession No. NM_003256.2).

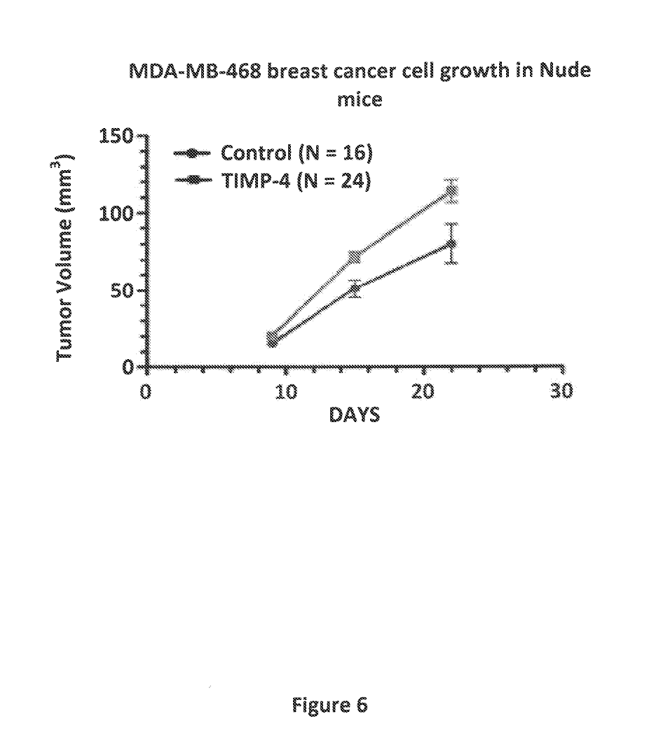

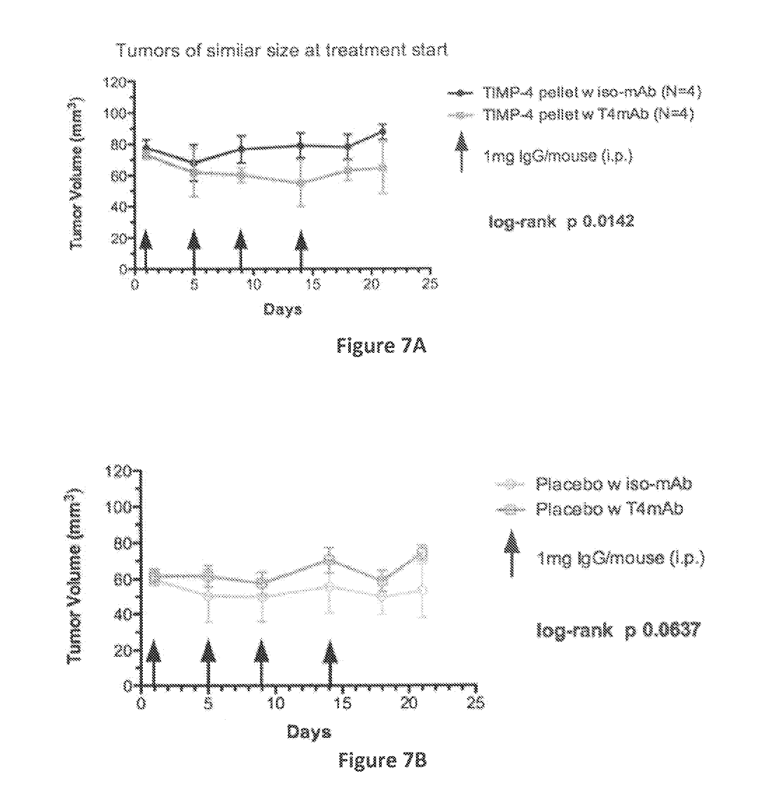

Inguinal mammary fat pads in nude mice were implanted with slow-release pellets (Innovative Research of America, Fla.) containing TIMP-4 to elevate its levels. Two weeks later, MDA-MB-468 cells were mixed with Matrigel™ (LDEV and phenol red free, BD Biosciences) and injected at the same site as an orthotopic xenograft. Differences in tumor size and growth rate were observed in the mice implanted with TIMP-4 pellets as compared to animals with placebo pellets (FIG. 6). Tumor-bearing mice from both the groups shown were then randomly divided in half and treated by i.p. injection with 1 mg IgG of TIMP-4 mAb (clone 18:4-7) or a matched isotype control IgG2bk (BioXCell, N.H.), in sterile azide-free solution, administered twice weekly for 2 weeks. In mice with elevated TIMP-4 levels, dosing of the TIMP-4 mAb caused an immediate and significant reduction in tumor size that was sustained post-treatment (FIG. 7A). The antibody had no effect on animals without elevated TIMP-4 (FIG. 7B). In tumors dissected 2 weeks after treatment and stained for pAkt^{Ser473}, a relative reduction in activated Akt was observed in tumors excised from mice treated with TIMP-4 mAb.

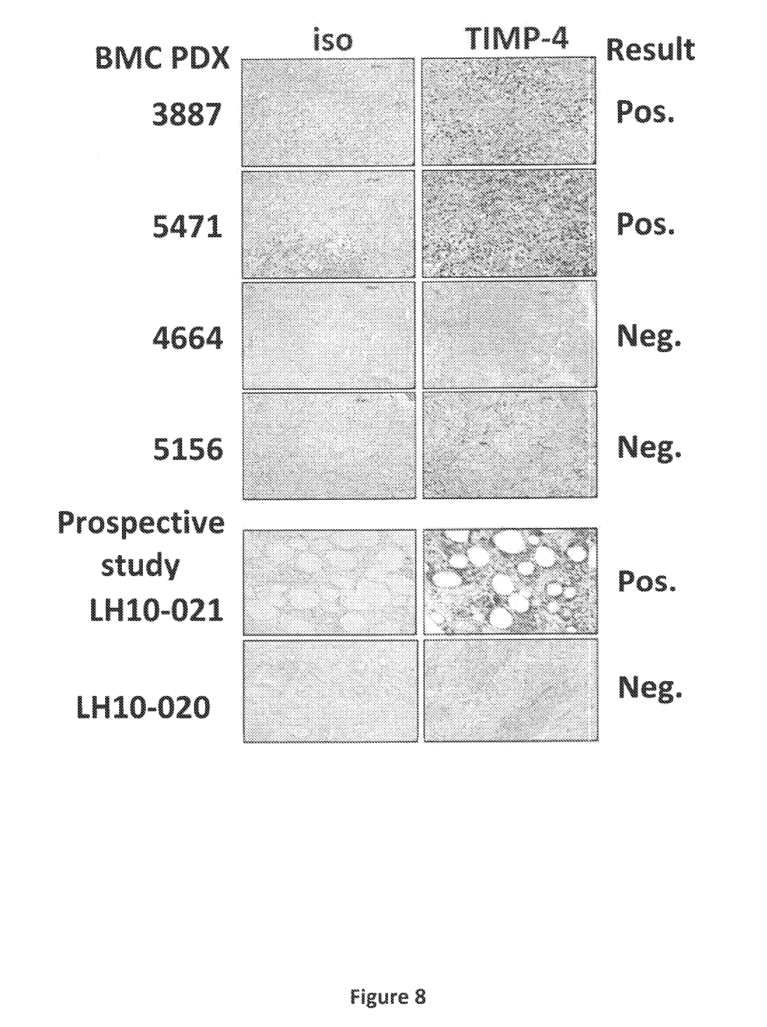

In addition to the above, an immunohistochemical analysis of multiple lines of a patient-derived xenograft (pdx) model was performed. The immunohistochemical analysis defined a set of 12 TNBC among the pdx tumors that were TIMP-4 positive or TIMP-4 negative (FIG. 8). These specific pdx may be used to evaluate the efficacy of TIMP-4 mAb alone or in combination with selected chemotherapy.

Together, the instant results: 1) demonstrate that that TIMP-4 offers a simple marker to stratify patients with the most aggressive TBNC for clinical treatment; 2) explain why only a subgroup of TNBC patients experience rapid relapses and dismal outcomes, as a result of TIMP-4 activating the PI3K/AKT pathway; and 3) provide a strategy of targeted immunotherapy to treat this subgroup of TNBC patients by attacking a pathologic mechanism that acts in parallel to the Her-2 pathway. By depleting elevated levels of TIMP-4 in the tumor microenvironment that can sustain the growth and survival of TNBC cells, including in small tumors otherwise thought to be curable, a TIMP-4 targeted mAb therapy would prevent recurrence, progression and early deaths in a group of breast cancer patients that remain poorly managed in the clinic.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of treating or inhibiting a cancer in a subject comprising administering an antibody or fragment thereof immunologically specific for tissue inhibitor of metalloproteinase-4 (TIMP-4) to said subject, wherein the tumor or surrounding tissue expresses TIMP-4.

2. The method of claim 1, wherein said cancer is breast cancer, ovarian cancer, brain cancer, or esophageal cancer.

3. The method of claim 1, wherein said cancer is breast cancer negative for estrogen receptor (ER), progesterone receptor (PR), and HER-2/neu.

4. The method of claim 1, further comprising the administration of at least one chemotherapeutic agent.

5. The method of claim 1, further comprising the administration of at least one biological agent.

6. The method of claim 1, further comprising resecting a tumor.

7. The method of claim 1, further comprising administering radiation therapy.

8. The method of claim 1, wherein said antibody or fragment thereof is a monoclonal antibody.

9. A method of treating, inhibiting, and/or preventing cardiovascular disease in a subject comprising administering an antibody or fragment thereof immunologically specific for tissue inhibitor of metalloproteinase-4 (TIMP-4) to said subject.

10. The method of claim 9, wherein said cardiovascular disease is a myocardial infarction.

11. A method of inhibiting the progression of a non-invasive cancer to an invasive cancer in a subject, said method comprising administering an antibody or fragment thereof immunologically specific for tissue inhibitor of metalloproteinase-4 (TIMP-4) to said subject.

12. The method of claim 11, wherein said cancer is breast cancer or prostate cancer.

13. The method of claim 11, wherein said cancer is breast cancer negative for estrogen receptor (ER), progesterone receptor (PR), and HER-2/neu.

14. The method of claim 11, further comprising the administration of at least one chemotherapeutic agent.

15. The method of claim 14, wherein said chemotherapeutic agent is an anthracycline.

16. The method of claim 11, wherein said antibody or fragment thereof is a monoclonal antibody.

17. A method of detecting cancer or an increased risk of invasiveness of a cancer in a subject, said method comprising detecting TIMP-4 in a biological sample obtained from a subject,
wherein an increase in TIMP-4 in the biological sample compared to a corresponding biological sample in a normal subject is indicative of cancer or an increased risk for cancer invasiveness in said subject.

18. The method of claim 17, wherein said cancer is breast cancer negative for estrogen receptor (ER), progesterone receptor (PR), and HER-2/neu.
